(19) United States
(12) Patent Application Publication
(10) Pub. No.: US 2015/0313489 A1
(43) Pub. Date: Nov. 5, 2015

(54) Wearable Device and Method for Assessing Cardiac Wellness by Analyzing High Definition EKG Signals

(71) Applicant: KENERGY, INC., Mequon, WI (US)

(72) Inventors: Cherik T. Bulkes, Sussex, WI (US); Mary Kay Norby, Menomonee Falls, WI (US)

(73) Assignee: KENERGY, INC., Mequon, WI (US)

(21) Appl. No.: 14/267,225

(22) Filed: May 1, 2014

(51) Int. Cl. A61B 5/04; G06F 19/00

(52) U.S. Cl. CPC A61B 5/04012; G06F 19/322

(57) ABSTRACT

A method for assessing cardiac wellness in a human utilizes high definition EKG signals to detect, analyze and compare waveform feature signatures at rest and during recovery from exertion. The parameters measured and displayed include beat-to-beat heart rate, heart rate variance and rate of recovery, which are indicators of the underlying physiological state. Combining these capabilities in a wearable device enables direct feedback to the user regarding overall fitness and physiologic response to therapeutic or behavioral interventions.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field Of The Invention

The present invention relates generally to the field of electrocardiography, and more specifically to the processing of high definition skin-surface cardiac signals for the purpose of assessing cardiac wellness in a personal wearable device.

2. Description of the Related Art

Personal monitoring of physiologic parameters is becoming increasingly widespread as consumers assume a more active role in monitoring and managing their own health. Whether the goal is to improve fitness at a very high level (e.g. athletic training), to monitor physical improvement during rehabilitation (e.g. following a heart attack), or as a prophylactic measure to ward off chronic disease and improve general health, the frequent and timely feedback that wearable monitors can provide is essential for enabling users to assess how physical activity and lifestyle choices, such as rest and use of stimulants, can affect overall physical health and wellness.

Specifically in the case of cardiac assessment, testing and monitoring is typically administered by a clinician in a clinical setting. This approach is costly and generally not covered by health insurance unless symptoms indicative of cardiac disease are present. Clinical testing requires stressing a patient beyond normal levels of exertion, which may or may not yield useful data about their cardiac health, and may put them at risk for injury. The sporadic nature of testing under these conditions provides limited feedback to the patient regarding the positive or negative effects of their lifestyle habits and/or therapeutic interventions.

In addition, new schools of thought are emerging within the cardiology profession, which indicate that traditional measures of cardiac activity, as derived from a typical electrocardiogram (EKG), provide an incomplete view of cardiac wellness. (Cipra, B., A healthy heart is a fractal heart, SIAM News, Vol. 36, No. 7, 2003); (Schlegel, T., et al., Real-time 12-lead high frequency QRS electrocardiography for enhanced detection of myocardial ischemia and coronary artery disease. Mayo Clinic Proc., Vol. 79, pp. 339-350, 2004); (Tragardh, E. & Schlegel, T., High-frequency QRS electrocardiogram, Clinical Physiology and Functional Imaging, Vol. 27, pp. 197-204, 2007). The heart is part of a larger feedback system and the signals it generates are far more dynamic and complex than previously recognized. (Oida, E., et al., Tone entropy analysis on cardiac recovery after dynamic exercise, Journal of Applied Physiology, Vol. 82, No. 6, pp. 1794-1801, 1997).

Recent trends are directed away from managing disease and toward promoting healthier lifestyles, a paradigm which relies on accurate self-monitoring, direct feedback, and timely intervention. This is evidenced by the rapid growth of the personal monitoring device market, which includes e.g. blood pressure monitors, pulse oximeters, glucose monitors, heart rate meters, activity monitors and personal EKG devices. A key feature of all these devices is that they are small, easy to operate, and portable enough to be worn and used routinely by the average consumer. Many of these devices also take advantage of the ubiquitous nature of application-enabled personal mobile computing device (PMCD) platforms (such as smart phones or tablet computers) for user interface functions, analysis, display and communication with remote servers.

Although a number of wearable consumer EKG devices are currently available that utilize PMCD platforms to acquire and display information, many of them require an over-read center for clinical interpretation before providing feedback to the user. This results in significantly delayed feedback (up to 24 hours) and expensive over-read fees. These devices also offer no comparative information regarding changes in cardiac activity before and after exertion. In addition, these systems rely on traditional low resolution EKG acquisition technology, which greatly limits their ability to discriminate true EKG signatures from noise. This requires use of extensive filtering, which further reduces and distorts signal content, limiting potential for reliable signal characterization and signature analysis.

### Electromagnetic Interference (EMI)

Traditional EKG systems monitor and/or record human cardiac conditions and activity by using a number of separately located individual electrodes attached to the skin surface to detect subcutaneous physio-electric signals. A composite of both subcutaneous physiological signals and environmental EMI are present on the human skin-surface. For medical monitoring, only the internal physiological signals are of interest; the external signals are classified as electromagnetic interference (EMI) and may significantly limit or reduce the ability to perform signature analysis.

External EMI on the human skin-surface originate from a variety of sources, including line source wiring, fluorescent lights, broadcasting services, appliances, medical instrumentation, computers, and electrical machinery. In the frequency band of interest (˜0.1 Hz thru ˜5 kHz) these signals can range beyond several millivolts/meter, whereas skin-surface physiological signal details are typically on the order of a few microvolts/meter. These numbers present potentially a very unfavorable Signal-to-Noise-Ratio (SNR), making it difficult to discern the smallest details in the internal physiological signals. This poor SNR limits the usefulness of current EKG sensing techniques.

### Filtering

In order to control noise in the signal, traditional EKG techniques typically employ multi-order low pass filtering, with a 40 Hz to 100 Hz upper frequency limit, while often requiring line frequency (50 Hz/60 Hz) notch filters. This limited bandwidth precludes analysis of the higher frequency EKG signal and limit temporal accuracy which are essential to the present invention.

Recent advancements in signal acquisition techniques have made it possible to sense physiological signals in their pristine form while avoiding error sources arising from electrical noise, signal amplitude variations, DC drift, and filtering (Ref. Denker, et al., U.S. Pat. No. 8,366,628). As they relate to this application, signals sampled using these techniques are hereinafter referred to as High Definition EKG and are defined as follows:
1) High bandwidth: bandwidth equal to or greater than 500 Hz and sampled at a rate at least two times the highest frequency of interest,
2) High resolution: digital signal samples having a resoultion of at least 10 bits or 60 dB,
3) Signal to noise ratio (SNR): better than 50 dB, and
4) Temporal accuracy: better than 100 parts per million. Traditional methods for analyzing EKG signals are based on either spectral and/or time domain analysis, however both of these methods present limitations in signature analysis.

### Spectral Analysis

Spectral analysis is harmonic decomposition of a dynamic waveform into its component frequencies and their relative amplitudes. In the case of electrocardiographic signals, the low frequency (less than 30 Hz) signals occupy nearly all of the dynamic range of a typical amplifier. Therefore, the usefulness of spectral analysis is generally limited to the lower frequency bands making it less suitable for detecting subtle details associated with the QRS complex and many rhythm disturbances, which tend to carry signature elements above 30 Hz. In addition, the presence of EMI and/or muscle artifact within traditional EKG signals can easily result in unfavorable signal to noise ratios, masking signals of interest.

### Time Domain Analysis

When high levels of EMI and line noise are present, time domain analysis is often employed to mitigate these sources of interference. For example, in order to filter out 50 Hz and 60 Hz line source noise, the upper frequency limit is usually less than 50 Hz, eliminating high frequency components. Even when notch filters are used, phase relationships will be affected. For example, introducing a notch filter to remove 45-65 Hz will introduce phase shifts over a range of at least 22 Hz to 130 Hz. Since signature information relevant to QRS waveforms and rhythm abnormality detection is contained in these bands, this type of filtering is not suitable for analyzing high definition EKG signals.

In view of the foregoing discussion, there is currently no method available that enables a consumer to accurately monitor and trend subtle changes in cardiac activity without involving a clinician. To be most beneficial, such a method would be provided in a wearable system and take advantage of the ubiquitous nature of PMCD platforms with or without remote server systems to acquire, analyze, display and store information. It would also be capable of providing direct feedback regarding positive or negative changes in cardiac activity that may result from therapeutic or behavioral interventions, such as medications, routine exercise or other lifestyle adjustments (e.g. smoking cessation, changing sleep habits or use of stimulants).

If implemented with the use of a remote server this can be used to facilitate global trending, however current smart phone and tablet platforms continue to increase in processing and storage capability, allowing for implementation with or without use of a remote server. The present invention provides such a method and introduces improvements over existing consumer EKG systems by utilizing high definition EKG signals to improve accuracy and enable direct user feedback regarding cardiac wellness.

### SUMMARY OF THE INVENTION

A method for assessing cardiac wellness uses signature analysis of high definition skin-surface EKG signals to measure beat-to-beat variance and rate of recovery in sequential EKG data samples. A wearable device comprises electrodes and an acquisition module which can contain the necessary functional components and algorithms for signal acquisition, signal analysis, presentation, data storage and data retrieval. Other embodiments can utilize wireless communications with an application-enabled PMCD (e.g. smart phone or tablet computer) to house specific functional components of the system, such as analysis, storage, retrieval and data and result presentation. Yet another embodiment can utilize a remote server for analysis, storage and retrieval to enable comparative trending and analysis.

In a preferred embodiment of this invention, the algorithms and display functions reside within a personal mobile computing device, such as a smart phone or tablet computer, and data is stored on the device as well as stored on a remote server to enable trending in comparison with other users and or groups.

In another embodiment of this invention, the algorithms, display functions and data storage reside within the acquisition module.

In other embodiments of this invention, the algorithms, display functions and data storage reside on any one of a number of combinations of acquisition module, personal mobile computing device, and remote server.

In one embodiment of this invention, the electrodes are integrated into a fixed array.

In another embodiment of this invention, the electrodes are individually applied to the skin.

### BRIEF DESCRIPTION OF THE DRAWINGS

FIG. 1 is a functional system block diagram of a device according to the present invention;

FIG. 2 is a functional system block diagram of an alternative device according to the present invention;

FIG. 3 is a functional system block diagram of a further embodiment of a device according to the present invention;

FIG. 4 is a block diagram of a resting EKG analysis algorithm;

FIG. 5 is a block diagram of a recovery EKG analysis algorithm;

FIG. 6 depicts one representative electrode array pattern;

FIG. 7 depicts an alternative electrode array pattern; and

FIG. 8 depicts another alternative electrode array pattern.

### DETAILED DESCRIPTION OF THE INVENTION

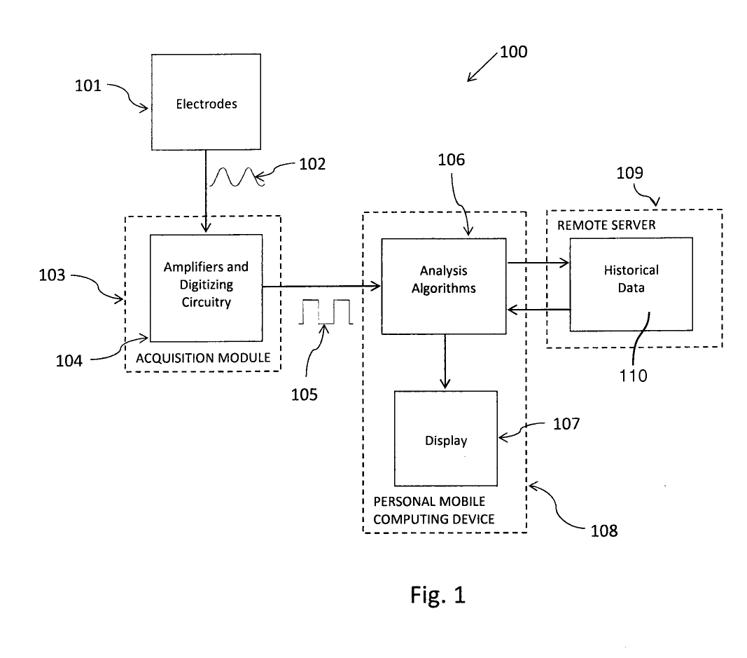

FIG. 1 is a schematic diagram of a method for analyzing high definition skin-surface EKG signals in a wearable device. The overall system 100 comprises a plurality of electrodes 101 which sense analog cardiac signals 102 that are transferred to an acquisition module 103. Multiple channels can be acquired to improve accuracy and/or optimize specific feature detection through redundancy. A channel typically requires a pair of electrodes 101. The acquisition module 103 is further comprised of circuitry 104 which amplifies and digitizes analog signals from the electrodes 101 to produce digital high definition EKG signals 105. Specifically, the acquisition module 103 has a bandwidth equal to or greater than 500 Hz and samples the analog cardiac signals 102 at a rate that is at least two times the highest frequency of interest, e.g. at a rate of 1.0 kHz. The samples are digitized into high resolution digital signal samples each having resolution of at least 10 bits. The acquisition module 103 provides a digital high definition EKG signal 105 that has a signal to noise ratio (SNR) that is better than 50 dB and a temporal accuracy that is better than 100 parts per million.

An analysis module 106 executes analysis algorithms that process the high definition EKG signals 105 to measure characteristic temporal and spatial features of the signal that are indicative of cardiac wellness. The results are displayed to the user. In this particular embodiment of the invention, the analysis module 106 and display device 107 reside on a personal mobile computing device 108 such as a smart phone or tablet computer, and data storage is done in a storage device 110 on a remote server 109 for later retrieval.

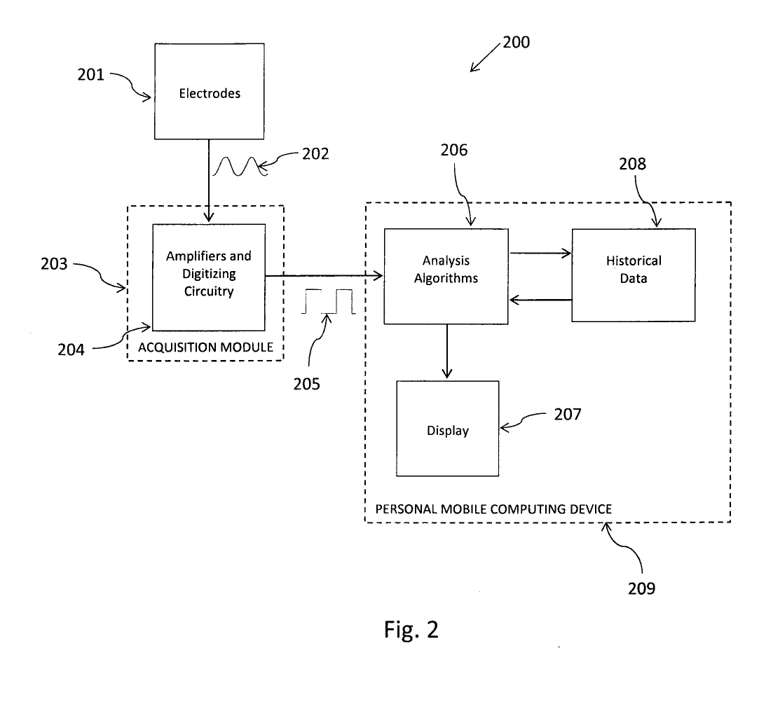

FIG. 2 is a schematic diagram of another embodiment of this invention in which the overall system 200 comprises a plurality of electrodes 201 which sense analog cardiac signals 202 that are transferred to an acquisition module 203. Multiple channels can be acquired to improve accuracy and/or optimize specific feature detection through redundancy. A channel typically requires a pair of electrodes. The acquisition module 203 is further comprised of circuitry 204 which amplifies and digitizes analog signals from the electrodes 201 to produce digital high definition EKG signals 205. The acquisition module 203 may be a microcomputer based device that is configured to be worn by the human whose heart is being monitored. An analysis module 206 executes analysis algorithms that process the high definition EKG signal 205 to measure characteristic temporal and spatial features of the signal that are indicative of cardiac wellness. This particular embodiment of the invention, the analysis module 206, display device 207 and data storage device 208 are all components of a personal mobile computing device 209, such as a smart phone or tablet computer.

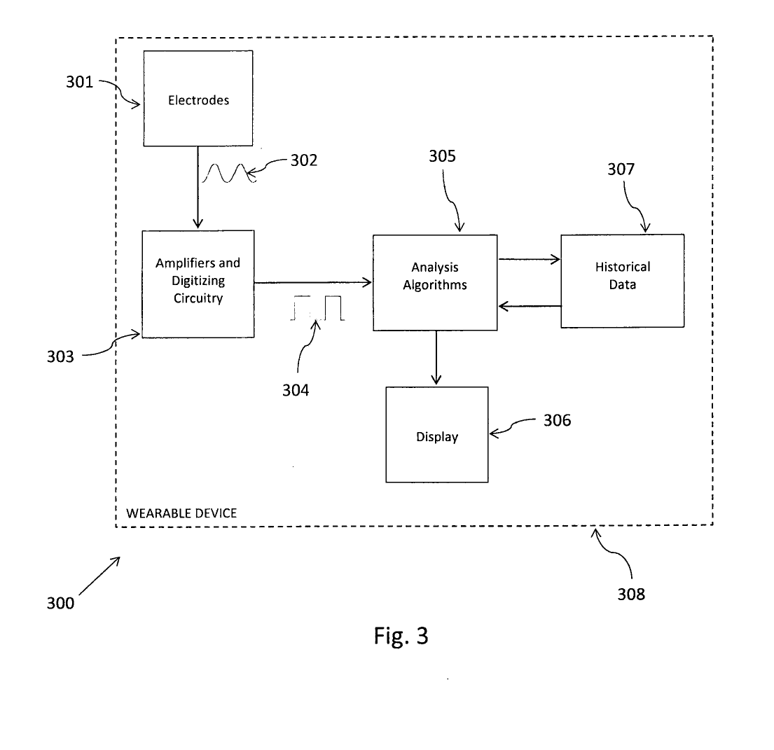

Another embodiment of the invention is shown in FIG. 3. In this embodiment the overall system 300 is comprised of a single wearable device 308 that integrates the sensing electrodes 301, amplifier and digitizing circuitry of a data acquisition module 303, an analysis module 305, display device 306, and data storage device 307.

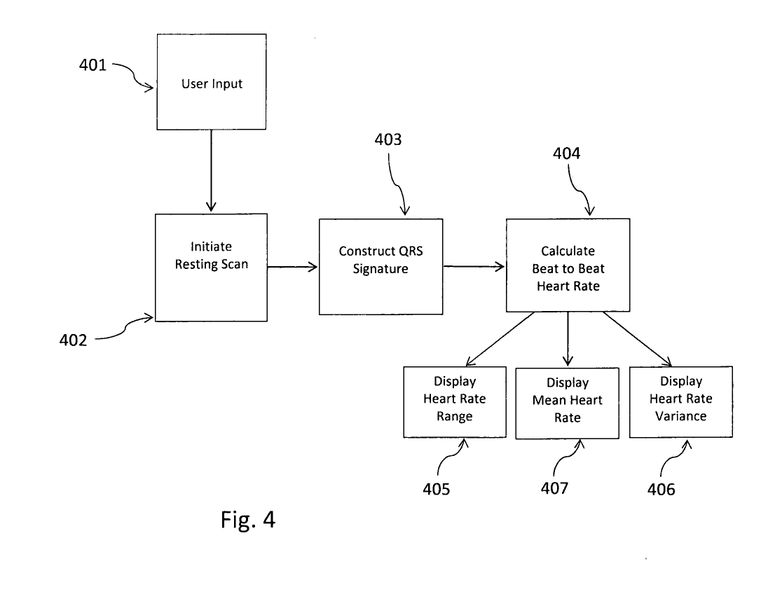

FIG. 4 is a schematic diagram of a representative measurement for assessing cardiac wellness from high definition EKG signals. User input 401 initiates a resting scan 402 of e.g. 10 to 100 seconds in length. This scan is used to determine EKG baseline parameters for later reference in the recovery analysis. Because temporal resolution is preserved in the high definition EKG signals, a waveform feature signature (e.g. of the QRS complex, P-wave or T-wave) 403 may be detected that allows beat-to-beat measurement 404 of heart rate with a higher degree of accuracy than is possible using traditional EKG methods, which have difficulty discriminating electromagnetic interference and other muscle activity from the actual cardiac signal of interest. This beat-to-beat variance is displayed as a range of heat rate 405 over the duration of the scan as well as a percentage of variance 406 from the median heart rate value 407. The heart rate variance is a function of the underlying physiological state, and tracking and trending this variance against activity levels and other physiological parameters (e.g. glucose levels) can provide insight as to cardiac wellness.

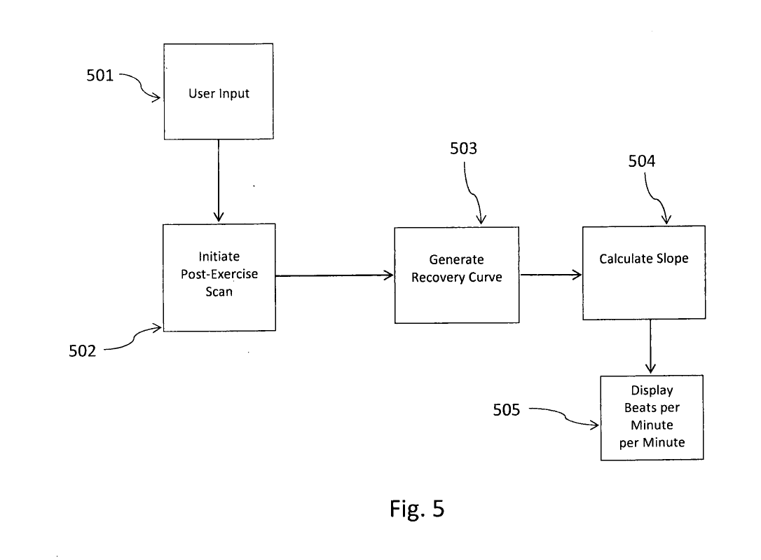

Referring to FIG. 5, signals may also be analyzed for dynamic recovery measurements immediately following exertion. In this case, user input 501 initiates a post-exercise scan 502 that is e.g. 30 to 120 seconds in length. A plot of the beat-to-beat heart rate measurements over time during this period produces a recovery curve 503. Due to the fact that high definition EKG preserves the amplitude and temporal resolution of the original signals, secondary signals, such as variances caused by respiratory motion, can be removed, allowing for calculation of a recovery rate 504 which can be measured and displayed in beats per minute per minute 505. In an otherwise healthy system, this recovery rate provides a measure of overall system fitness.

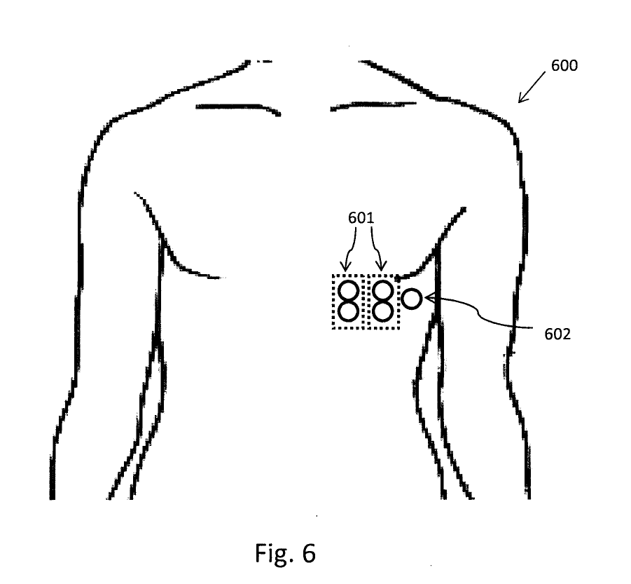

FIG. 6 illustrates representative electrode pattern 600 for capturing signals appropriate for assessing cardiac wellness using the methods described. Electrodes are arranged in pairs 601 in an (N X M)+1 array where 1<N<x and 1<M<y with practical numbers being x=2 and y=2 to 8. One additional electrode 602 acts an electronics drain return to the skin-surface to prevent or reduce charge build up resulting from the electrode-skin interface.

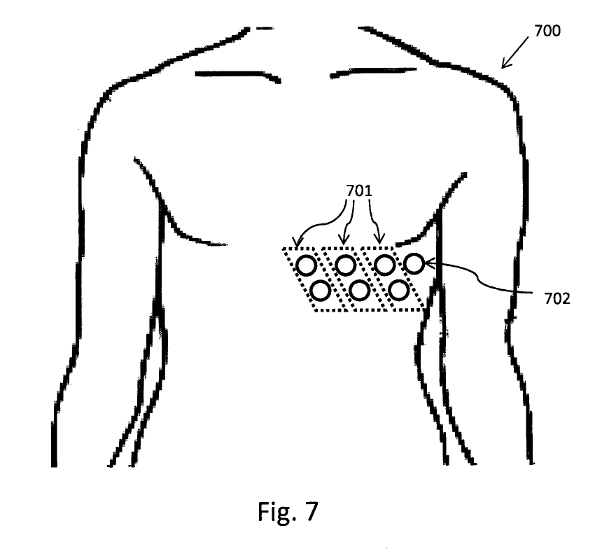

The rows may be arranged in a rectangular grid as in FIG. 6 or in a staggered array 700 as in FIG. 7. Electrodes are arranged in pairs 701 in an (N X M)+1 array where 1<N<x and 1<M<y with practical numbers being x=2 and y=2 to 8. One additional electrode 702 acts an electronics drain return to the skin-surface to prevent or reduce charge build up resulting from the electrode-skin interface.

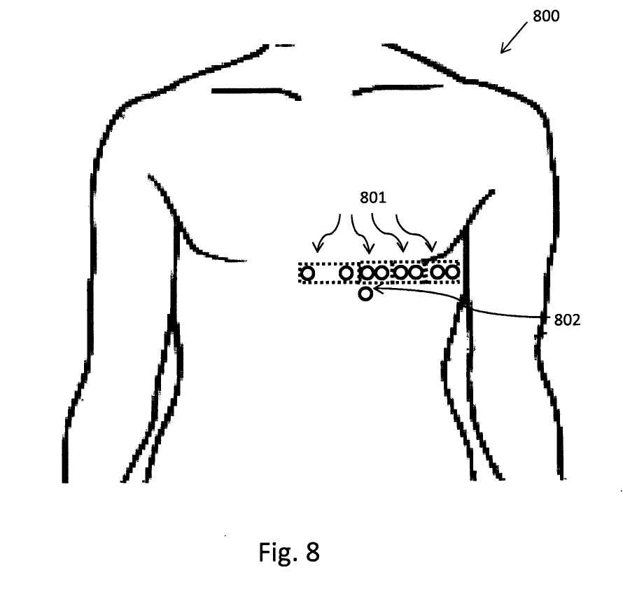

Other electrode pattern layouts and positions can be used to optimize signal acquisition for specific cardiac conditions, such as the presence of suspected or known arrhythmias. FIG. 8 is an exemplary electrode pattern layout 800 that is optimized for capturing p-wave activity in which at least one electrode pair 801 spans both sides of the sternum. One additional electrode 802 acts an electronics drain return to the skin-surface to prevent or reduce charge build up resulting from the electrode-skin interface.

FIGS. 6, 7 and 8 are illustrative of typical configurations, but should not be considered limiting, as multiple additional configurations are possible.

The foregoing description was primarily directed to preferred embodiments of the invention. Although some attention was given to alternatives within the scope of the invention, it is anticipated that one skilled in the art will likely realize additional alternatives that are now apparent from disclosure of the embodiments of the invention. Accordingly, the scope of the invention should be determined from the following claims and not limited by the above disclosure.

We claim:

1. A method for assessing cardiac wellness of a human using differential analysis of High Definition EKG features at rest and during recovery from exertion comprising:
while the human is resting, acquiring a set of resting data samples of high definition EKG signals from at least one pair of electrodes located on an external skin surface of the human;
detecting at least one predefined waveform feature signature in the set of resting data samples;
calculating a beat-to-beat heart rate, a median heart rate, and a variance in heart rate over a duration of the set of resting data samples;
after a period of physical exertion of the human, acquiring a set of recovery data samples of high definition EKG signals from at least one pair of electrodes located on an external skin surface of the human;
analyzing the set of recovery data samples to remove secondary signals;
from the set of recovery data samples, constructing a recovery curve of heart rate over time;
calculating a recovery rate, thereby producing recovery rate calculations;
displaying results of said recovery rate calculations to a user, such results being indicative of degree of cardiac wellness;
storing the results of said recovery rate calculations; and
storing the set of resting data samples and the set of recovery data samples for later retrieval and serial analysis.

2. The method of claim 1 wherein the high definition EKG signals possess characteristics of:
a bandwidth equal to or greater than 500 Hz,
a resolution of digital signal samples having at least 10 bits,
a signal to noise ratio better than 50 dB, and
a temporal accuracy better than 100 parts per million.

3. The method of claim 2 wherein acquiring a set of resting data samples, detecting at least one predefined waveform, calculating beat-to-beat heart rate, mean heart rate and variance in heart rate, acquiring a set of recovery data samples, analyzing the set of recovery data samples, constructing a recovery curve, calculating a recovery rate, displaying the results, storing the results, and storing the set of resting data samples and the set of recovery data samples are performed by circuitry contained within a single module that is wearable by the human.

4. The method of claim 1 wherein acquiring a set of resting data samples and acquiring a set of recovery data samples are performed by circuitry contained within a module that is wearable by the human.

5. The method of claim 4 wherein detecting at least one predefined waveform, calculating beat-to-beat heart rate, mean heart rate and variance in heart rate, analyzing the set of recovery data samples, constructing a recovery curve, calculating a recovery rate, and displaying the results are performed by a personal mobile computing device.

6. The method of claim 5 wherein the personal mobile computing device comprises one of a smart phone and a tablet computer.

7. The method of claim 6 wherein storing the results of said recovery rate calculations, and storing the set of resting data samples and the set of recovery data samples are performed by the personal mobile computing device.

8. The method of claim 6 wherein storing the results of said recovery rate calculations, and storing the set of resting data samples and the set of recovery data samples are performed by a remote server.

9. A system for assessing cardiac wellness of a human comprising:
at least one pair of electrodes adapted to be placed on an external skin surface of the human;
data acquisition module for acquiring a set of resting data samples of high definition EKG signals from the at least one pair of electrodes while the human is resting, and for acquiring a set of recovery data samples of high definition EKG signals from the at least one pair of electrodes after a period of physical exertion by the human;
an analysis module for:
a) detecting at least one predefined waveform feature signature in the set of resting data samples,
b) calculating a beat-to-beat heart rate, a median heart rate and a variance in heart rate over duration of the set of resting data samples,
c) analyzing the set of recovery data samples to remove secondary signals,
d) from the set of recovery data samples, constructing a recovery curve of heart rate over time, and
e) calculating a recovery rate thereby producing recovery rate calculations;
a display device for displaying results of said recovery rate calculations to a user, such results being indicative of degree of cardiac wellness; and
a storage device to storing the results of said recovery rate calculations, the set of resting data samples and the set of recovery data samples.

10. The system of claim 9 wherein the at least one pair of electrodes comprises a flexible electrode array of M rows and N columns of electrodes and a drain electrode, where M and N are greater than or equal to one.

11. The system of claim 10 wherein the flexible electrode array is integrated into the data acquisition module.

12. The system of claim 10 wherein the flexible electrode array is connected to the data acquisition module by conductor wires.

13. The system of claim 9 wherein the data acquisition module is configured to be worn on the human.

14. The system of claim 13 wherein the at least one pair of electrodes comprises individual electrode patches connected to the data acquisition module by conductor wires.

15. The system of claim 13 wherein the analysis module, the display device, and the storage device are contained within a personal mobile computing device.

16. The system of claim 15 wherein the personal mobile computing device comprises one of a smart phone and a tablet computer.

17. The system of claim 9 wherein the analysis module, and the display device are contained within a personal mobile computing device; and the storage device is contained within a server that is remote from the personal mobile computing device.

18. The system of claim 9 wherein a user interface and the display device are contained within a personal mobile computing device; and the analysis module and the storage device are contained within a remote server.
